(19) United States
(12) Patent Application Publication
(10) Pub. No.: US 2013/0324827 A1
(43) Pub. Date: Dec. 5, 2013

(54) Atraumtic Clamp-Type Electrode Contact

(71) Applicant: Dennis Burkett, Malvern, PA (US)

(72) Inventors: Gary H. Reitnauer, Quakertown, PA (US); Dennis Burkett, Malvern, PA (US)

(21) Appl. No.: 13/907,395

(22) Filed: May 31, 2013

Related U.S. Application Data
(60) Provisional application No. 61/654,078, filed on May 31, 2012.

(51) Int. Cl. A61D 99/00; A61B 5/0478; A61N 1/04; A61B 5/0408

(52) U.S. Cl. CPC A61D 99/00; A61B 5/0408; A61B 5/0478; A61N 1/0472
USPC 600/386

(57) ABSTRACT

A clamp-type electrode contact for physiological use on animals including a first clamping member, a second clamping member, a connecting lead member, and a spring means. A spring engaging member disposed between first and second clamping members and connecting lead member may be included. Spring means is adapted for being placed in a first position when clamping members are in a closed position, and being placed in a second position when clamping members are in an open position. At least one inner side of clamping members includes engagement grooves formed along the inner side. Engagement grooves preferably form a crosshatch pattern, and each groove preferably has a rounded radius and is sufficiently shallow as not to introduce any epidermal or vascular trauma. First and second clamping members are generally aligned with one another whereby inner side of first clamping member and inner side of second clamping member face one another.

### RELATED APPLICATIONS

This patent application is related to provisional patent application Ser. No. 61/654,078 entitled “Atraumatic Clamp-Type Electrode Contact” filed on May 31, 2012, priority from which is hereby claimed.

### FIELD OF THE INVENTION

The present invention relates generally to medical devices, systems, and the like for physiological use. More specifically, the present invention relates to an atraumatic, clamp-type electrode contact for physiological use on animals.

### BACKGROUND OF THE INVENTION

Many medical devices, systems, and the like for physiological use on humans and animals are known in the art. The present invention relates to a non-slip, clamp-type electrode contact for physiological use, such as for electrocardiogram (ECG or EKG), electroencephalogram (EEG), defibrillation, cardiac pacers, stimulation, and any medical or electrophysiological equipment that requires electrodes to interface with an animal patient, such as but not limited to dogs and cats. One current practice in this field is to use a self-adhesive button electrode, which is not easily movable once in place and which requires the animal's hair to be shaved for the electrode to make contact with the skin. Some veterinary technicians or practitioners solve these problems by using a standard alligator clip electrode contact, such as the one shown in FIG. 12, which has serrated teeth on the contact jaws. The problem with this practice is that the serrated teeth have pinch points, cause skin trauma and sometimes cause vascular damage. Therefore, some practitioners flatten the serrated teeth with pliers or file them off. However, the absence of teeth causes the electrode contact to slip or come off the animal during monitoring. Also, the alligator clip electrode contact has pinch points and a center pivot point design, which causes non-uniform pressure points, which in turn interferes with transmission of signal.

Information relevant to attempts to address these problems can be found in U.S. Patent Application No. 2009/0062636, published to Muz; U.S. Pat. No. 4,781,197, issued to Fukuda; U.S. Pat. No. 4,612,936, issued to Yamaguchi et al.; U.S. Pat. No. 6,006,130, issued to Higo et al.; U.S. Pat. No. 2,611,368, issued to Pecora; U.S. Pat. No. 5,511,546, issued to Hon; U.S. Pat. No. 7,018,345, issued to Mori et al.; U.S. Pat. No. 5,209,679, issued to Quinlan; and U.S. Pat. No. 6,799,076, issued to Gelb et al.

Therefore, there is a need for an electrode contact that is easily movable and does not require the animal's hair to be shaved, yet provides a non-slip, superior connection to the animal without causing skin or vascular trauma.

The present invention solves the problems identified above by adapting a crocodile clip design, such as that shown in U.S. Pat. No. 1,999,613, issued to Mueller et al. (as opposed to an alligator clip design as shown in FIG. 12) with features that provide an atraumatic (causing no trauma) yet non-slip and superior connection to the animal that is easily movable and does not require the animal's hair to be shaved. Advantages over existing products on the market include: absence of serrated teeth or other features which could cause skin and vascular trauma; absence of adhesives, which enables use on unshaved skin and easy movability; crosshatch pattern of grooves on the clamping surfaces provides an interlace design that prevents lateral and longitudinal slippage; rounded crosshatch grooves and rounded edges provide a non-slip grip of the skin without trauma, even on unshaved animals; crocodile clamp-like electrode contact provides consistent contact area with uniform pressure points and larger contact area than current alligator clamp-like electrode contact designs, with no pinch points.

The Applicant is unaware of inventions or patents, taken either singly or in combination, which are seen to describe the instant invention as claimed.

### SUMMARY OF THE INVENTION

The present invention is an atraumatic, non-slip, clamp-type electrode contact for physiological use, such as for electrocardiogram (ECG or EKG), electroencephalogram (EEG), defibrillation, cardiac pacers, stimulation, and any medical or electrophysiological equipment that requires electrodes to interface with an animal patient, such as but not limited to dogs and cats.

In one embodiment, the clamp-type electrode contact for physiological use on animals includes a first clamping member, a second clamping member, a connecting lead member adapted for being secured to a physiological monitoring, stimulation or testing device, and a spring means. The electrode contact may further include a spring engaging member disposed between the first and second clamping members and the connecting lead member.

The first clamping member and second clamping member form a pair of jaws structure for securing or clamping a body tissue of an animal patient. Preferably, the pivot point of the electrode contact is located toward the rear end of the electrode contact where the first clamping member and second clamping member are pivotally secured to one another.

The first clamping member includes a front end, a rear end, an intermediate portion disposed between the front end and rear end, a first side, a second side, an outer side, and an inner side. The inner side of the first clamping member includes a front edge, a first side edge, a second side edge, and a plurality of engagement grooves formed along the inner side of the first clamping member.

The second clamping member includes a front end, a rear end, an intermediate portion disposed between the front end and rear end, a first side, a second side, an outer side, and an inner side. The inner side of the second clamping member includes a front edge, a first side edge, a second side edge, and a plurality of engagement grooves formed along the inner side of the second clamping member.

The first clamping member and second clamping member are generally aligned with one another whereby the inner side of the first clamping member and the inner side of the second clamping member face one another. The first clamping member and second clamping member are adapted for being placed together alternatively in a closed position and an open position with respect to one another relative to a clamp arrangement. The first clamping member and second clamping member are adapted for clamping a body tissue or member of an animal when in the closed position.

The connecting lead member is adapted for being secured to a physiological monitoring, stimulation or testing device.

When included, the spring engaging member is preferably disposed between the first clamping member and connecting lead member and disposed between the second clamping member and connecting lead member. The spring engaging member includes a front end, a rear end, and an intermediate portion disposed between the front end and rear end of the spring engaging member.

The front end of the spring engaging member is located about the rear end of the clamping member and about the rear end of the second clamping member. The rear end of the spring engaging member is located about the connecting lead member. The spring engaging member is adapted for engaging with a spring means whereby the first clamping member and second clamping member are movable from the closed position to the open position and vice versa with respect to one another.

The spring means is adapted for being placed in a first position when the first clamping member and second clamping member are in the closed position. The spring means is adapted for being placed in a second position when the first clamping member and second clamping member are in the open position wherein the first position is an unloaded or extended position and second position is a loaded or compressed position, or wherein said first position is a loaded or compressed position and the second position is an unloaded or extended position.

Preferably, a pivot point is located about the rear end of the spring engaging member.

Preferably, the engagement grooves of the first clamping member and second clamping member form a crosshatch pattern disposed between the first side edge and second side edge of the clamping member; and each of a majority of the engagement grooves has a rounded radius and is sufficiently shallow as not to cause any skin or vascular trauma.

Preferably, the edges of the clamping member are generally smooth and/or rounded.

### BRIEF DESCRIPTION OF THE DRAWINGS

FIG. 1 is a left side perspective view of a clamp-type electrode contact according to the present invention, wherein a pair of clamping members or sections of the electrode contact is in a closed position;

FIG. 2 is an environmental, left side perspective view of the clamp-type electrode contact of FIG. 1, wherein the pair of clamping members or sections is in an open position;

FIG. 3 is an environmental, front elevation view of the clamp-type electrode contact of FIG. 1, wherein the pair of clamping members or sections is in an open position;

FIG. 4 is a front elevation view of the clamp-type electrode contact of FIG. 1, with emphasis on the spring means, wherein the pair of clamping members or sections is in the closed position;

FIG. 5 is a rear perspective view of the clamp-type electrode contact of FIG. 1, wherein the pair of clamping members or sections is in the closed position;

FIG. 6 is a top view of the clamp-type electrode contact of FIG. 1;

FIG. 7 is a top view of the clamp-type electrode contact of FIG. 1, wherein the pair of clamping members or sections is in the closed position;

FIG. 8 is a bottom view of the clamp-type electrode contact of FIG. 1;

FIG. 9 is a perspective view of a clamping member or section of a clamp-type electrode contact according to the present invention, wherein an inner side of the clamping member or section comprises a plurality of engagement grooves forming a crosshatch pattern disposed between a first side edge and a second side edge of the inner side of the clamping member or section;

FIG. 10 is a side view of a first clamping member or section and a second clamping member or section of a clamp-type electrode contact according to the present invention, wherein an inner side of the first clamping member or section and the inner side of the second clamping member or section face one another and wherein engagement grooves along the inner side of the first clamping member or section are in alignment with the corresponding engagement grooves along the inner side of the second clamping member or section when the clamping members or sections are in, or about in, the closed position;

FIG. 11 is an environmental view of the clamp-type electrode contact of FIG. 1, wherein the electrode contact and other electrode contacts that are exactly the same as, or substantially similar to, the electrode contact are secured to an animal patient; and

FIG. 12 is a perspective view of a standard alligator clip electrode contact in the prior art.

It should be understood that the Figures are illustrative of one embodiment of the invention and should not be construed to limit the scope of the present invention in any way.

### DETAILED DESCRIPTION OF THE PREFERRED EMBODIMENTS

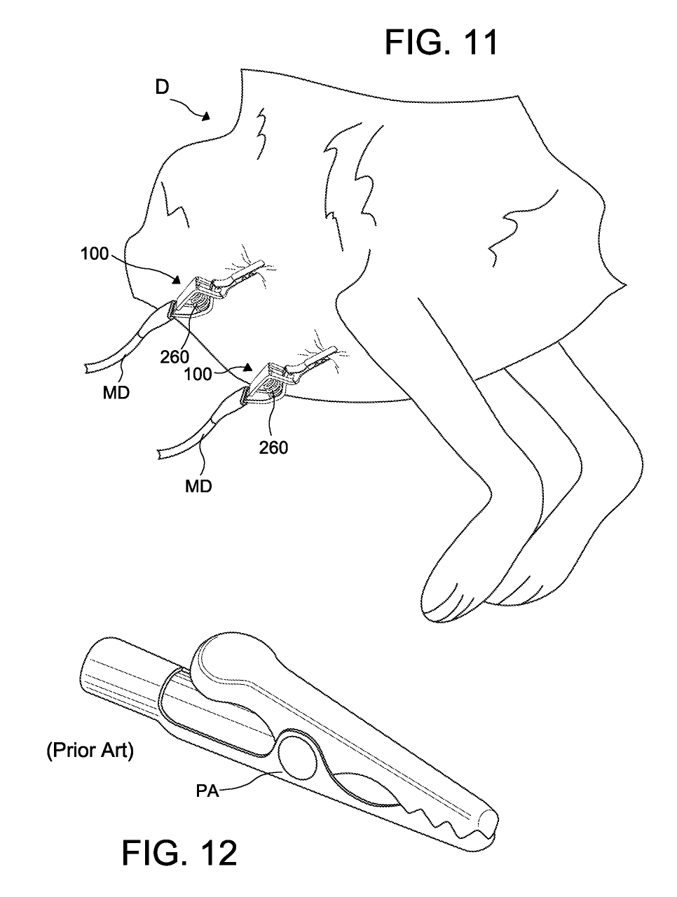

The present invention is an atraumatic, non-slip, clamp-type electrode contact 100 for physiological use, such as for electrocardiogram (ECG or EKG), electroencephalogram (EEG), defibrillation, cardiac pacers, stimulation, and any medical or electrophysiological equipment that requires electrodes to interface with an animal patient, such as but not limited to dogs D (see FIG. 11), cats, and other household pets.

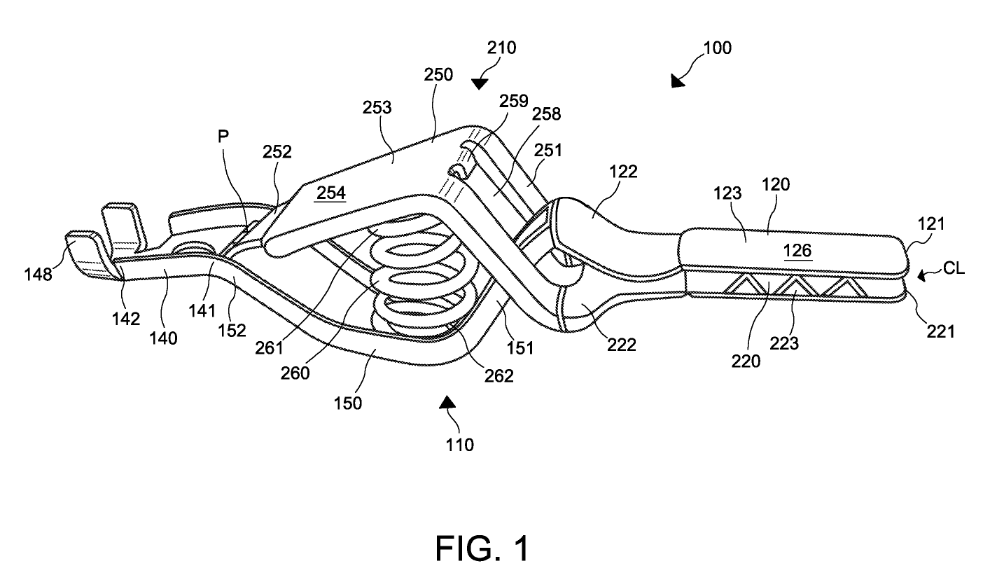

Referring to FIG. 1, an embodiment and non-limiting example of a clamp-type electrode contact 100 for physiological use on animals includes a first, elongated clamping member 110, a second, elongated clamping member 210, and a spring means 260. The first, elongated clamping member 110 and second, elongated clamping member 210 are configured and arranged such that their applicable sections form a pair of jaws structure for securing or clamping a body tissue of an animal patient and a housing-like structure for securing and engaging the spring means 260. Preferably, the pivot point P of the clamp-type electrode contact 100 is located toward the rear end of the clamp-type electrode contact 100 where the first, elongated clamping member 110 and second, elongated clamping member 210 are pivotally secured to one another.

The first, elongated clamping member 110 includes a first clamping section 120, a connecting lead section 140, and a first spring engaging section 150 disposed between the first clamping section 120 and connecting lead section 140.

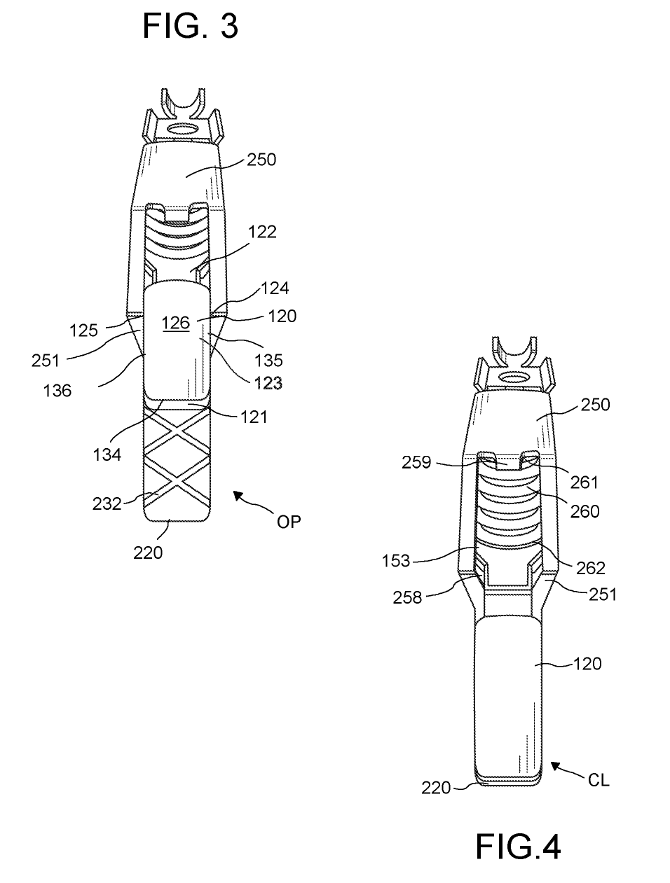

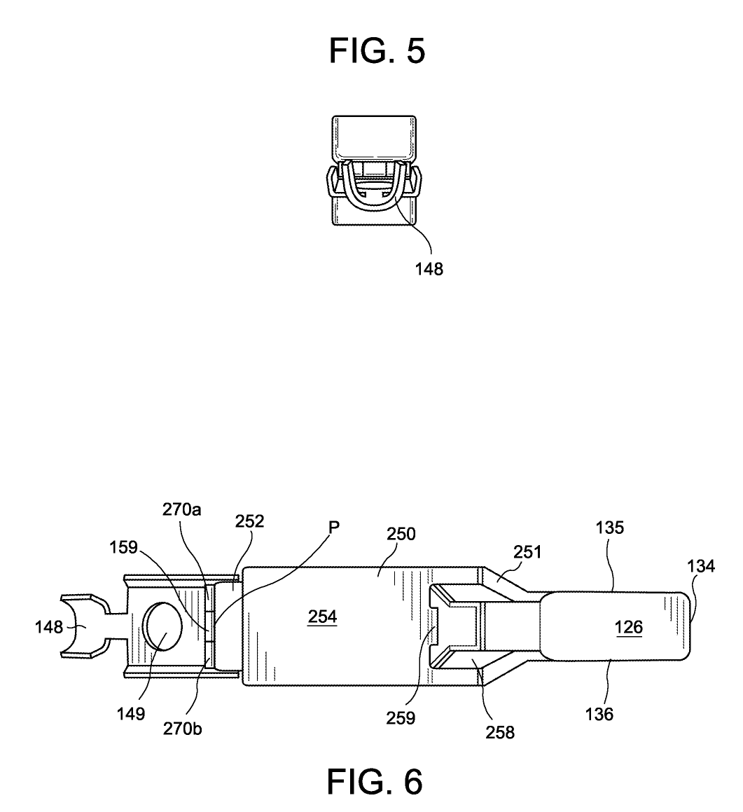

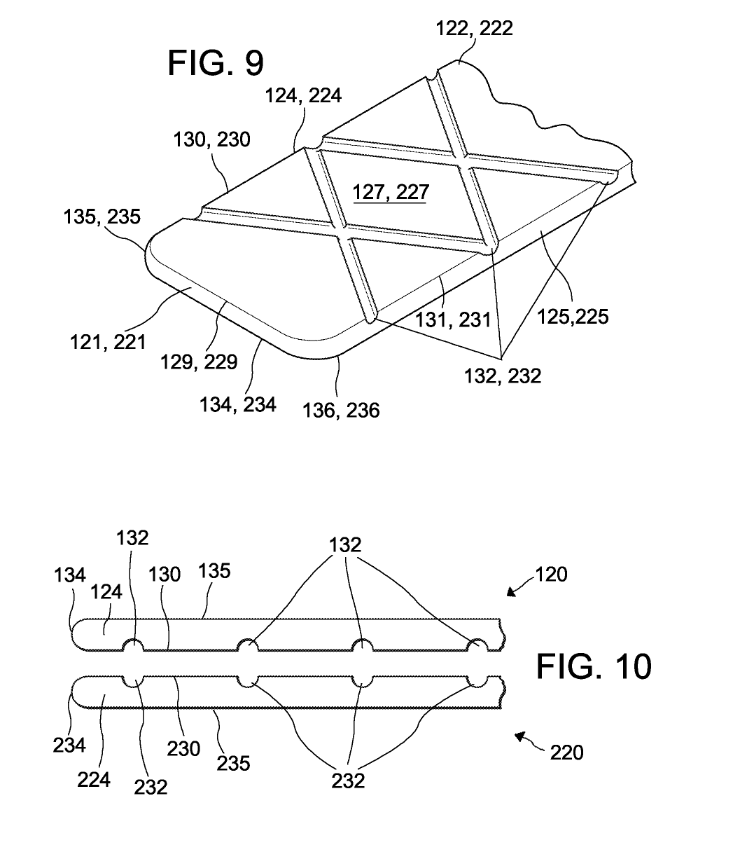

As best shown in FIG. 3, the first clamping section 120, or “upper jaw” of the pair of jaws structure, includes a front end 121, a rear end 122, an intermediate portion 123 disposed between the front end 121 and rear end 122, a first side 124, a second side 125, an outer side 126, and an inner side 127 (see FIG. 9). Preferably, the first clamping section 120 is elongated and has a generally rectangular configuration wherein the first side 124 and second side 125 are opposite and generally parallel to one another and wherein the outer side 126 and inner side 127 are opposite and generally parallel to one another. Preferably, both of the outer side 126 and inner side 127 (see FIG. 9) are generally planar or flat.

Referring now to FIGS. 9 and 10, the inner side 127 includes a front edge 129, a first side edge 130, a second side edge 131, and a plurality of engagement grooves 132 formed along the inner side 127 to prevent slippage of the electrode contact during monitoring and animal movement. The outer side 126 (see FIG. 3) includes a front edge 134, a first side edge 135, and a second side edge 136. Preferably, the edges 129, 130, 131, 134, 135, 136 and engagement grooves 132 are generally smooth and/or rounded to prevent or minimize skin abrasions. Preferably and as a non-limiting example, the engagement grooves 132 form a crosshatch pattern disposed between the first side edge 130 and second side edge 131 of the inner side 127, as best shown in FIG. 9. Preferably, each engagement groove 132 of the crosshatch pattern has a rounded radius and is shallow enough as not to introduce any epidermal or vascular trauma yet still maintaining a non-slip grip of the skin and superior skin contact even on an unshaved animal. The crosshatch pattern provides an interlace design that grips the skin in multiple directions so as to avoid lateral and longitudinal slippage.

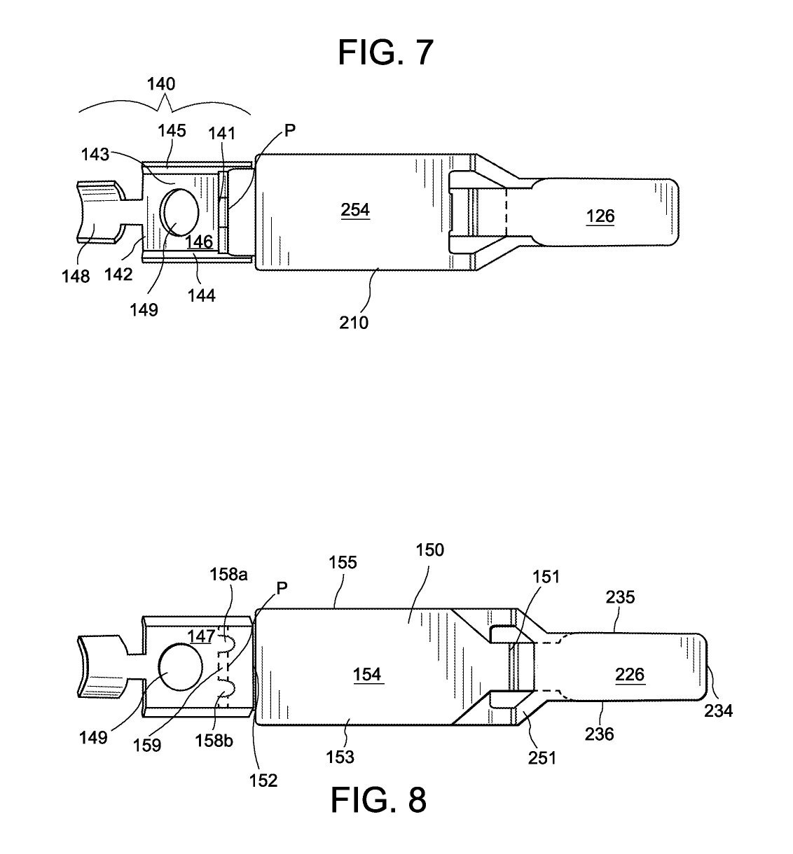

FIG. 7 depicts the connecting lead section 140 which is adapted for being secured to a physiological monitoring device MD (seen in FIG. 11). The connecting lead section 140 includes a front end 141, a rear end 142, and an intermediate portion 143 disposed between the front end 141 and rear end 142. Preferably, the connecting lead section 140 further includes a first side 144, a second side 145, a top side 146, a bottom side 147 (see FIG. 8), a lead-supporting member 148 located about the rear end 142 and a hole 149 substantially centrally located at the intermediate portion 143. The hole 149 is dimensioned and configured for receiving a screw or the like that secures a conduction lead or the like to the connecting lead section 140.

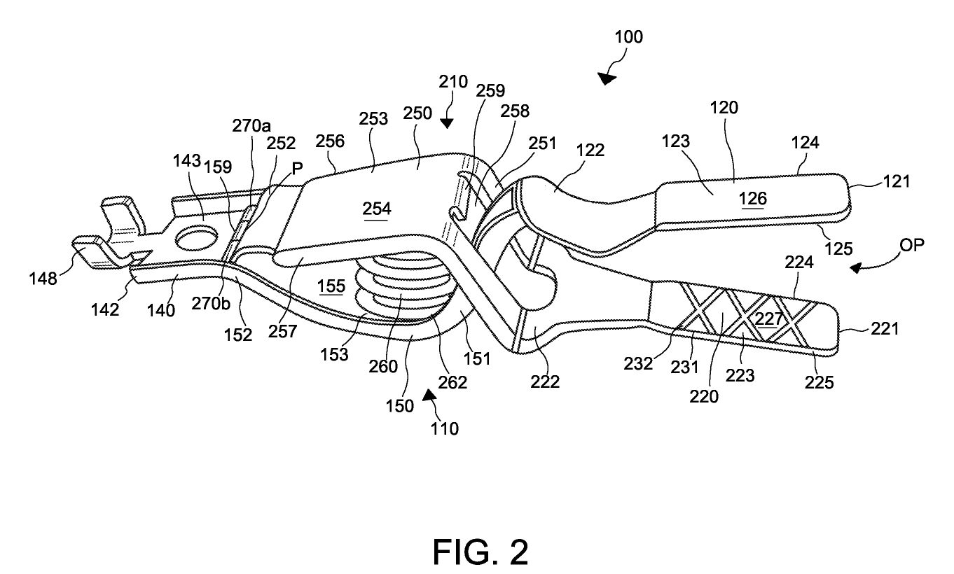

FIG. 8 details the first spring engaging section 150, or the lower portion of the housing-like structure, which includes a front end 151, a rear end 152, an intermediate portion 153 disposed between the front end 151 and rear end 152, an outer side 154, and an inner side 155 (see FIG. 2). Preferably, the first spring engaging section 150 further includes first and second pivot point slots 158a, 158b and a pivot point extension 159 located about the rear end 152 to form or define a pivot point P with the corresponding structure or section of the second, elongated clamping member 210. As best shown in FIG. 8, the pivot point extension 159 is located intermediate of the pivot point slots 158a, 158b, and extends upwardly at about a ninety degree angle. Preferably, the first spring engaging section 150 has a generally U-shaped configuration as shown in FIG. 1.

Referring again to FIG. 1, the front end 151 of the first spring engaging section 150 is located about the rear end 122 of the first clamping section 120, and extends downwardly and rearwardly from the rear end 122 of the first clamping section 120. The rear end 152 of the first spring engaging section 150 is located about the front end 141 of the connecting lead section 140, and extends downwardly and forwardly from the front end 141 of the connecting lead section 140. Referring now to FIG. 2, the intermediate portion 153 of the first spring engaging section 150 is adapted for engaging with the second end 262 of the spring means 260.

The second, elongated clamping member 210 includes a second clamping section 220 and a second spring engaging section 250.

Preferably and as best shown in FIGS. 1 and 2, the second clamping section 220, or “lower jaw” of the pair of jaws structure, is substantially similar to the first clamping section 120, or “upper jaw” of the pair of jaws structure. The second clamping section 220 includes a front end 221, a rear end 222, an intermediate portion 223 disposed between the front end 221 and rear end 222, a first side 224, a second side 225, an outer side 226 (see FIG. 8), and an inner side 227. Preferably, the second clamping section 220 is elongated and has a generally rectangular configuration wherein the first side 224 and second side 225 are opposite and generally parallel to one another and wherein the outer side 226 (see FIG. 8) and inner side 227 are opposite and generally parallel to one another. Preferably, both of the outer side 226 (see FIG. 8) and inner side 227 are generally planar or flat.

Referring now to FIG. 9, as preferably substantially similar to the inner side 127 of the first clamping section 120, the inner side 227 of the second clamping section 220 includes a front edge 229, a first side edge 230, a second side edge 231, and a plurality of engagement grooves 232 formed along the inner side 227 to prevent slippage of the electrode contact during monitoring and animal movement. As shown in FIGS. 8 and 9, the outer side 226 includes a front edge 234, a first side edge 235, and a second side edge 236. Preferably, the edges 229, 230, 231, 234, 235, 236 and engagement grooves 232 are generally smooth and/or rounded to prevent or minimize skin abrasions. Preferably and as a non-limiting example, the engagement grooves 232 form a crosshatch pattern disposed between the first side edge 230 and second side edge 231 of the inner side 227. Referring now to FIG. 10, preferably, each engagement groove 232 of the crosshatch pattern has a rounded radius and is shallow enough as not to introduce any epidermal or vascular trauma yet still maintaining a non-slip grip of the skin and superior skin contact even on an unshaved animal. The crosshatch pattern provides an interlace design that grips the skin in multiple directions so as to avoid lateral and longitudinal slippage. For general veterinary work with dogs, cats, rabbits, weasels, rats and mice, and electrode contacts of copper, grooves having a depth in the range 0.006 inches to 0.010 inches are optimally shallow enough so as not to introduce any epidermal or vascular trauma yet still maintaining a non-slip grip of the skin and superior skin contact. It should be understood that the optimal range of groove depth may vary according to the animal patient and the particular materials and design of the electrode contact according to the invention.

Referring to FIGS. 2 and 6, the second spring engaging section 250, or the upper portion of the housing-like structure, includes a front end 251, a rear end 252, an intermediate portion 253 disposed between the front end 251 and rear end 252, an outer side 254, an inner side 255 (not shown, but corresponding to an equivalent inner side 155 of the first spring engaging section 150), and a cutout 258 about the front end 251 of the second spring engaging section 250. Preferably, the second spring engaging section 250 further includes a first side 256, a second side 257 and a front end extension 259 that help to retain and secure the spring means 260 within the housing-like structure, and also first and second pivot point extensions 270a, 270b located about the rear end 252 to form or define the pivot point P (see FIG. 8) with the corresponding pivot point slots 158a, 158b of the first, elongated clamping member 110. Referring to FIGS. 2, 6 and 8, pivot point extensions 270a, 270b extend through the corresponding pivot point slots 158a, 158b and are secured about the rear end 152 of the first spring engaging section 150 on the outer side 154. Pivot point extension 159 is in close proximity to the rear end 252 of the second spring engaging section 250 on the outer side 254. As best shown in FIGS. 1 and 2, in general, the pivoting movement of the second, elongated clamping member 210 relative to the first, elongated clamping member 110 is in the vertical direction, with little or no pivoting movement in the lateral direction. Preferably, the second spring engaging section 250 has a generally inverted V-shaped configuration as shown in FIGS. 1 and 2.

Still referring to FIG. 2, the front end 251 of the second spring engaging section 250 is located about the rear end 222 of the second clamping section 220, and extends upwardly and rearwardly from the rear end 222 of the second clamping section 220. The rear end 252 of the second spring engaging section 250 is located about the front end 141 (see FIG. 1) of the connecting lead section 140, and extends upwardly and forwardly from the front end 141 of the connecting lead section 140. Referring to both FIGS. 2, 3 and 4, the intermediate portion 253 of the second spring engaging section 250 is adapted for engaging with the first end 261 of the spring means 260.

As best shown in FIGS. 1 and 2, the cutout 258 is sufficiently dimensioned for housing the front end 151 of the first spring engaging section 150 and for allowing the front end 151 of the first spring engaging section 150 to be moved within the cutout 258 when the first clamping section 120 and the second clamping section 220 are moved with respect to one another from a closed position CL to an open position OP and vice versa.

The first, elongated clamping member 110 and second, elongated clamping member 210 are arranged with respect to one another such that the first clamping section 120 and second clamping section 220 are generally aligned, preferably substantially aligned, with one another and are adapted for securing or clamping a body tissue or member, such as the skin, of an animal D (see FIG. 11) when in the closed position CL, whereby the first clamping section 120 is positioned above the second clamping section 220, whereby the inner side 127 (see FIGS. 2, 9 and 10) of the first clamping section 120 and the inner side 227 of the second clamping section 220 face one another, whereby the second spring engaging section 250 is positioned above the first spring engaging section 150, and whereby the inner side 155 of the first spring engaging section 150 and the inner side 255 of the second spring engaging section 250 face one another.

Referring now to FIGS. 9 and 10, when the inner side 127 of the first clamping section 120 and the inner side 227 of the second clamping section 220 face one another when the clamping members 110, 210 are in, or about in, the closed position CL (see FIGS. 1 and 4), it is preferred that the engagement grooves 132 are in alignment with the corresponding engagement grooves 232.

Referring now to FIG. 4, the spring means 260 includes a first end 261 and a second end 262. Preferably, the spring means 260 is any spring, such as a coiled spring, a compression spring, a torsion spring, a leaf spring, a polyurethane spring, a rubber spring, or device that is known to one of ordinary skill in the art that can accomplish the intended purpose of the present invention when the first clamping section 120 (see FIG. 1) and the second clamping section 220 are moved with respect to one another from a closed position CL to an open position OP (see FIGS. 2 and 3) and vice versa.

Referring now to FIGS. 2 and 4 in conjunction, the first end 261 of the spring means 260 is adapted for being secured to or engaging with the intermediate portion 253 of the second spring engaging section 250, while the second end 262 of the spring means 260 is adapted for being secured to or engaging with the intermediate portion 153 of the first spring engaging section 150. Preferably, the spring means 260 is in an unloaded or extended position when the first clamping section 120 and second clamping section 220 are in the closed position CL. Preferably, the spring means 260 is in a loaded or compressed position when the first clamping section 120 and second clamping section 220 are in the open position OP as shown in FIGS. 2-3. Preferably, the first clamping section 120 and second clamping section 220 are moved from the closed position CL to the open position OP when a user presses the intermediate portion 253 of the second spring engaging section 250 downward toward the intermediate portion 153 of the first spring engaging section 150.

Preferably, as shown in FIGS. 1 and 2, the clamp-type electrode contact 100 further includes a pivot point P located about the rear ends 152, 252 of the first spring engaging section 150 and second spring engaging section 250. One advantage of an electrode contact 100 of the preferred embodiment, having the pivot point P at the rear of the electrode contact 100, is that it provides a substantially parallel contact area with uniform pressure points, in contrast to an electrode contact of the alligator type shown in FIG. 12, with centrally located pivot point PA. Another advantage of an electrode contact of the preferred embodiment is that it has a larger contact area than that provided by an alligator type clip, with no pinch points.

The electrode contact 100 may be made or manufactured from any conductive material, such as but not limited to stainless steel, copper, steel, plated steel, aluminum, nickel, brass, gold, silver and even carbon-induced polymers, and any combination thereof, known to one of ordinary skill in the art. One non-limiting example would be for the electrode contact 100 to be electroplated in gold to produce a non-corrosive, totally bio-compatible electrode. Another non-limiting example would for the electrode contact 100 to be made or plated in at least 60 percent copper to produce an electrode contact with antimicrobial properties. Another non-limiting example would be for the electrode contact 100 to be made entirely from a non-magnetically reactive or non-metallic material, including a non-magnetically reactive spring means 260, to produce an electrode contact that is magnetically non-reactive, such as for use with Magnetic Resonance Imaging (MRI) or X-Ray equipment.

Other variations and embodiments of the electrode contact of the invention may be conceived by one skilled in the art. It is to be understood that the present invention is not limited to the embodiments described above or as shown in the attached figures, but encompasses any and all embodiments within the spirit of the invention.

What is claimed is:

1. A clamp-type electrode contact for physiological use on animals comprising:
a first clamping member comprising a front end, a rear end, an intermediate portion disposed between said front end and said rear end, a first side, a second side, an outer side, and an inner side,
wherein said inner side of said first clamping member comprises a front edge, a first side edge, and a second side edge;
a second clamping member comprising a front end, a rear end, an intermediate portion disposed between said front end and said rear end of said second clamping member, a first side, a second side, an outer side, and an inner side,
wherein said inner side of said second clamping member comprises a front edge, a first side edge, and a second side edge,
wherein at least one of said inner side of said first clamping member and said inner side of said second clamping member comprises a plurality of engagement grooves,
wherein at least one of said inner side of said first clamping member and said inner side of said second clamping member is generally planar,
wherein said first clamping member and said second clamping member are generally aligned with one another wherein said inner side of said first clamping member and said inner side of said second clamping member face one another,
wherein said first clamping member and said second clamping member are pivotally secured to one another,
wherein a pivot point of said clamp-type electrode contact is located toward a rear end of said clamp-type electrode contact, and
wherein said first clamping member and said second clamping member are adapted for being pivotally positioned alternatively in an open position and a closed position, and are adapted for clamping a body tissue of an animal patient when in said closed position;
spring means adapted for being in communication with said first clamping member and said second clamping member,
wherein said spring means is placed in a first position when said first clamping member and said second clamping member are in said closed position, and being placed in a second position when said first clamping member and said second clamping member are in said open position; and
a connecting lead member adapted for being secured to a physiological testing device.

2. The clamp-type electrode contact according to claim 1, wherein at least one of said first clamping member and said second clamping member is elongated.

3. The clamp-type electrode contact according to claim 2, wherein said elongated clamping member has a generally rectangular configuration wherein said first side and said second side are opposite and generally parallel to one another and wherein said outer side and said inner side are opposite and generally parallel to one another.

4. The clamp-type electrode contact according to claim 1, wherein said edges are rounded.

5. The clamp-type electrode contact according to claim 1, wherein said first clamping member and said second clamping member are substantially aligned with one another.

6. The clamp-type electrode contact according to claim 1, wherein said plurality of engagement grooves form a crosshatch pattern disposed between said first side edge and said second side edge of said inner side, and wherein each of a sufficient amount of said plurality of engagement grooves has a rounded radius and is sufficiently shallow as not to introduce any epidermal or vascular trauma.

7. The clamp-type electrode contact according to claim 6, wherein said inner side of said first clamping member comprises a first plurality of engagement grooves and said inner side of said second clamping member comprises a second plurality of engagement grooves, wherein said first plurality of engagement grooves are similar to said second plurality of engagement grooves, and wherein said first plurality of engagement grooves correspondingly align with said second plurality of engagement grooves when said first clamping member and said second clamping member are in said closed position.

8. The clamp-type electrode contact according to claim 1, further comprising a spring engaging member disposed between said first clamping member and said connecting lead member and disposed between said second clamping member and said connecting lead member,
wherein said spring engaging member comprises a front end, a rear end, and an intermediate portion disposed between said front end and said rear end of said spring engaging member,
wherein said front end of said spring engaging member is located about said rear end of said first clamping member and about said rear end of said second clamping member,
wherein said rear end of said spring engaging member is located about said connecting lead member, and
wherein said spring engaging member is adapted for engaging with said spring means whereby said first clamping member and said second clamping member are movable from said closed position to said open position and vice versa with respect to one another.

9. The clamp-type electrode contact according to claim 8, wherein said spring engaging member further comprises an upper side and a lower side, wherein said upper side is secured to and engages with a first end of said spring means, wherein said lower side is secured to and engages with a second end of said spring means, wherein said upper side has a generally U-shaped configuration, and wherein said lower side has a generally inverted V-shaped configuration.

10. A clamp-type electrode contact for physiological use on animals comprising:
a first clamping member comprising a front end, a rear end, an intermediate portion disposed between said front end and said rear end, a first side, a second side, an outer side, and an inner side,
wherein said inner side of said first clamping member comprises a front edge, a first side edge, and a second side edge;
a second clamping member comprising a front end, a rear end, an intermediate portion disposed between said front end and said rear end of said second clamping member, a first side, a second side, an outer side, and an inner side,
wherein said inner side of said second clamping member comprises a front edge, a first side edge, and a second side edge,
wherein at least one of said inner side of said first clamping member and said inner side of said second clamping member comprises a plurality of engagement grooves,
wherein said plurality of engagement grooves form a crosshatch pattern disposed between said first side edge and said second side edge of said inner side,
wherein each of a sufficient amount of said plurality of engagement grooves has a rounded radius and is sufficiently shallow as not to introduce any epidermal or vascular trauma,
wherein said first clamping member and said second clamping member are generally aligned with one another wherein said inner side of said first clamping member and said inner side of said second clamping member face one another,
wherein said first clamping member and said second clamping member are pivotally secured to one another, and
wherein said first clamping member and said second clamping member are adapted for being pivotally positioned alternatively in an open position and a closed position, and are adapted for clamping a body tissue of an animal patient when in said closed position;
spring means adapted for being in communication with said first clamping member and said second clamping member,
wherein said spring means is placed in a first position when said first clamping member and said second clamping member are in said closed position, and being placed in a second position when said first clamping member and said second clamping member are in said open position; and
a connecting lead member adapted for being secured to a physiological device.

11. The clamp-type electrode contact according to claim 10, wherein at least one of said first clamping member and said second clamping member is elongated.

12. The clamp-type electrode contact according to claim 11, wherein said elongated clamping member has a generally rectangular configuration wherein said first side and said second side are opposite and generally parallel to one another and wherein said outer side and said inner side are opposite and generally parallel to one another.

13. The clamp-type electrode contact according to claim 10, wherein said edges are rounded.

14. The clamp-type electrode contact according to claim 10, wherein said first clamping member and said second clamping member are substantially aligned with one another.

15. The clamp-type electrode contact according to claim 10, wherein said inner side of said first clamping member comprises a first plurality of engagement grooves and said inner side of said second clamping member comprises a second plurality of engagement grooves, wherein said first plurality of engagement grooves are similar to said second plurality of engagement grooves, and wherein said first plurality of engagement grooves correspondingly align with said second plurality of engagement grooves when said first clamping member and said second clamping member are in said closed position.

16. The clamp-type electrode contact according to claim 10, wherein a pivot point of said clamp-type electrode contact is located toward a rear end of said clamp-type electrode contact.

17. The clamp-type electrode contact according to claim 10, further comprising a spring engaging member disposed between said first clamping member and said connecting lead member and disposed between said second clamping member and said connecting lead member,
wherein said spring engaging member comprises a front end, a rear end, and an intermediate portion disposed between said front end and said rear end of said spring engaging member,
wherein said front end of said spring engaging member is located about said rear end of said first clamping member and about said rear end of said second clamping member,
wherein said rear end of said spring engaging member is located about said connecting lead member, and
wherein said spring engaging member is adapted for engaging with said spring means whereby said first clamping member and said second clamping member are movable from said closed position to said open position and vice versa with respect to one another.

18. The clamp-type electrode contact according to claim 17, wherein said spring engaging member further comprises an upper side and a lower side, wherein said upper side is secured to and engages with a first end of said spring means, wherein said lower side is secured to and engages with a second end of said spring means, wherein said upper side has a generally U-shaped configuration, and wherein said lower side has a generally inverted V-shaped configuration.

19. The clamp-type electrode contact according to claim 11,
wherein each of said first clamping member and said second clamping member is elongated,
wherein said first, elongated clamping member has a generally rectangular configuration wherein said first side and said second side are opposite and generally parallel to one another and wherein said outer side and said inner side are opposite and generally parallel to one another,
wherein said second, elongated clamping member has a generally rectangular configuration wherein said first side and said second side are opposite and generally parallel to one another and wherein said outer side and said inner side are opposite and generally parallel to one another,
wherein each of said inner side of said first clamping member and said inner side of said second clamping member is generally planar,
wherein said inner side of said first clamping member comprises a first plurality of engagement grooves and said inner side of said second clamping member comprises a second plurality of engagement grooves,
wherein said first plurality of engagement grooves are similar to said second plurality of engagement grooves,
wherein said first plurality of engagement grooves correspondingly align with said second plurality of engagement grooves when said first clamping member and said second clamping member are in said closed position, and
wherein a pivot point of said clamp-type electrode contact is located toward a rear end of said clamp-type electrode contact.

20. The clamp-type electrode contact according to claim 19, wherein said edges are rounded.

21. The clamp-type electrode contact according to claim 19, wherein said first clamping member and said second clamping member are substantially aligned with one another.

22. The clamp-type electrode contact according to claim 19, further comprising a spring engaging member disposed between said first clamping member and said connecting lead member and disposed between said second clamping member and said connecting lead member,
wherein said spring engaging member comprises a front end, a rear end, and an intermediate portion disposed between said front end and said rear end of said spring engaging member,
wherein said front end of said spring engaging member is located about said rear end of said first clamping member and about said rear end of said second clamping member,
wherein said rear end of said spring engaging member is located about said connecting lead member, and
wherein said spring engaging member is adapted for engaging with said spring means whereby said first clamping member and said second clamping member are movable from said closed position to said open position and vice versa with respect to one another.

23. The clamp-type electrode contact according to claim 22, wherein said spring engaging member further comprises an upper side and a lower side, wherein said upper side is secured to and engages with a first end of said spring means, wherein said lower side is secured to and engages with a second end of said spring means, wherein said upper side has a generally U-shaped configuration, and wherein said lower side has a generally inverted V-shaped configuration.

24. The clamp-type electrode contact according to claim 19, wherein said connecting lead section comprises a front end, a rear end, an intermediate portion disposed between said front end and said rear end, a first side, a second side, a top side, a bottom side, a hole located about said intermediate portion, and a lead-supporting member located about said rear end of said connecting lead section.
